(19) United States
(12) Patent Application Publication
(10) Pub. No.: US 2014/0135663 A1
(43) Pub. Date: May 15, 2014

(54) ULTRASONIC VIBRATION DEVICE

(71) Applicant: OLYMPUS CORPORATION, Tokyo (JP)

(72) Inventors: Tomoki FUNAKUBO, Tokyo (JP); Hiroshi TSURUTA, Kanagawa (JP)

(73) Assignee: OLYMPUS CORPORATION, Tokyo (JP)

(21) Appl. No.: 14/162,069

(22) Filed: Jan. 23, 2014

Related U.S. Application Data
(63) Continuation of application No. PCT/JP2012/067463, filed on Jul. 9, 2012.

(30) Foreign Application Priority Data
Aug. 5, 2011 (JP) 2011-172066

(51) Int. Cl. A61N 7/00; B06B 1/06

(52) U.S. Cl. CPC A61N 7/00; B06B 1/0603
USPC 601/2; 310/330

(57) ABSTRACT

Provided is an ultrasonic vibration device with which it is possible to efficiently remove fat by generating vibrations not only in the direction parallel to a probe but also in the direction orthogonal to the probe. Provided is an ultrasonic vibration device including a polygonal columnar elastic body formed of an elastic body; a piezoelectric element that is secured to a side surface of the polygonal columnar elastic body and that is polarized in a plate-thickness direction thereof; a rod-like contactor that is secured to an end portion of the polygonal columnar elastic body and that has a smaller diameter than the polygonal columnar elastic body; and a drive-pulse generating circuit that generates a bending vibration in the polygonal columnar elastic body by applying an AC voltage in the plate-thickness direction of the piezoelectric element, thus generating an ultrasonic vibration in the rod-like contactor.

### CROSS-REFERENCE TO RELATED APPLICATIONS

This is a continuation of International Application PCT/JP2012/067463, with an international filing date of Jul. 9, 2012, which is hereby incorporated by reference herein in its entirety. This application claims the benefit of Japanese Patent Application No. 2011-172066, the content of which is incorporated herein by reference.

### TECHNICAL FIELD

The present invention relates to an ultrasonic vibration device employing a piezoelectric element.

### BACKGROUND ART

In the related art, there are known devices for removing fat, an example of which is an ultrasonic vibration device for removing animal fat tissue disclosed in Patent Literature 1. With this device, a bolt-clamped Langevin transducer is employed as a transducer, fat is emulsified by means of vibrations at the distal end of a probe, and the emulsified fat is sucked from a through-hole at a center portion thereof.

### CITATION LIST

### Patent Literature

{PTL1} Japanese Examined Patent Application, Publication No. Hei 6-20462

### SUMMARY OF INVENTION

### Technical Problem

With the device disclosed in Patent Literature 1, vibrations can be excited only in the direction parallel to the probe, and for example, when employed in a procedure in which fat at a surface of the heart is removed without performing a thoracotomy, vibrations cannot be excited in a direction orthogonal to the probe, and thus, the fat cannot be removed efficiently.

The present invention provides an ultrasonic vibration device with which it is possible to efficiently remove fat by generating vibrations not only in the direction parallel to a probe but also in the direction orthogonal to the probe.

### Solution to Problem

The present invention employs an ultrasonic vibration device provided with a columnar member formed of an elastic body; a piezoelectric element that is secured to a side surface of the columnar member and that is polarized in a plate-thickness direction thereof; a rod-like member that is secured to an end portion of the columnar member and that has a smaller diameter than the columnar member; and a voltage applying portion that generates a bending vibration in the columnar member by applying an AC voltage in the plate-thickness direction of the piezoelectric element, thus generating an ultrasonic vibration in the rod-like member.

With the present invention, the bending vibration is generated in the columnar member formed of the elastic body by applying the AC voltage in the plate-thickness direction of the piezoelectric element by means of the voltage applying portion. This vibration is transmitted to the rod-like member, which is secured to the end portion of the columnar member, thus generating the ultrasonic vibration in the rod-like member. By inserting the rod-like member exhibiting such an ultrasonic vibration into, for example, a body cavity such as a pericardial cavity or the like, and by bringing the rod-like member into contact with fat adhered to an inner wall of the body cavity, it is possible to melt (emulsify) the fat by means of the ultrasonic vibration.

In this case, the bending vibration of the columnar member in the present invention, that is, the vibration transmitted to the rod-like member, is a vibration in the direction orthogonal to the axis of the rod-like member. Therefore, in the state in which the rod-like member is inserted into the body cavity, it is possible to melt fat by means of the ultrasonic vibration, not only at the distal-end surface of the rod-like member but also at a side surface thereof, and thus, it is possible to efficiently melt the fat adhered to the inner wall of the body cavity.

In the above-described invention, the columnar member may be a polygonal columnar member.

By employing such a configuration, the piezoelectric elements can be disposed at the four side surfaces of the polygonal columnar member, and it is possible to efficiently generate the bending vibration in the polygonal columnar member by means of these piezoelectric elements. By doing so, it is possible to intensify the vibration transmitted to the rod-like member, and thus, it is possible to efficiently melt the fat adhered to the inner wall of the body cavity. Note that it is possible to achieve a size reduction of the device by disposing the pair of the piezoelectric elements on the two opposing side surfaces of the columnar member.

In the above-described invention, the columnar member may be a pyramidal member whose a lateral cross-sectional area gradually decreases toward a connecting position with the rod-like member.

By employing such a configuration, it is possible to make the mechanical impedances of the rod-like member and the pyramidal member similar at the connecting position between the rod-like member and the pyramidal member. Because it is possible to achieve good matching of the mechanical impedances between the two members in this way, it is possible to efficiently transmit the vibrational energy of the pyramidal member to the rod-like member.

In addition, by employing the pyramidal member, it is possible to increase the lateral cross-sectional area of the pyramidal member at the proximal end thereof. By doing so, large surface areas can be ensured for the piezoelectric elements 12 to be secured to the side surfaces of the pyramidal member, and thus, the vibrational energy generated at the pyramidal member can be increased.

In addition, because it is possible to make the distal end of the pyramidal member smaller, it is possible to enhance the ease of insertion into a body cavity and so forth, thus improving the usability of the device.

The above-described invention may be provided with a pair of the piezoelectric elements that are disposed facing each other with the columnar member placed therebetween, and the pair of the piezoelectric elements may be disposed so that orientations of the polarizations thereof point in the same direction.

By employing such a configuration, by connecting the pair of piezoelectric elements facing each other with a lead, it is possible to make this pair of piezoelectric elements expand/contract in opposite phases to each other, and thus, it is possible to efficiently generate the bending vibration in the columnar member.

The above-described invention may be provided with a pair of the piezoelectric elements that are disposed facing each other with the columnar member placed therebetween, and the pair of the piezoelectric elements may be disposed so that orientations of the polarizations thereof point in opposite directions.

By employing such a configuration, without having to connect the pair of piezoelectric elements facing each other with a lead, it is possible to make this pair of piezoelectric elements expand/contract in opposite phases to each other, and it is possible to generate the bending vibration in the columnar member. By doing so, the number of leads for connecting the piezoelectric elements can be reduced.

The above-described invention may be provided with a plurality of the piezoelectric elements, and the plurality of piezoelectric elements may be disposed side-by-side in an axial direction of the columnar member so that the polarizations of the adjacent piezoelectric elements are oriented differently.

By employing such a configuration, it is possible to generate a bending vibration of an even higher-order mode in the columnar member. By doing so, it is possible to increase the number of antinodes in the rod-like member, that is, the positions at which the amplitude becomes the highest, and thus, it is possible to more efficiently melt the fat adhered to the inner wall of the body cavity.

The above-described invention may be provided with a case that accommodates the columnar member and a holding member that is provided between the case and the columnar member and that holds the columnar member at a node of the bending vibration.

By employing such a configuration, it is possible to hold the columnar member in the case by means of the holding member. By holding the columnar member at a node of the bending vibration in this way, it is possible to prevent the vibrational energy generated at the columnar member from escaping outside the case. By doing so, it is possible to efficiently generate the ultrasonic vibration in the rod-like member.

The above-described invention may be provided with a suction path for sucking tissue, provided inside the rod-like member and the columnar member.

By employing such a configuration, tissue melted by the ultrasonic vibration of the rod-like member (for example, emulsified-fat components) can be externally discharged via the suction path.

The above-described invention may be provided with a liquid-supplying path provided inside the rod-like member and the columnar member.

By employing such a configuration, liquid for facilitating propagation of the ultrasonic vibration of the rod-like member to a biological subject, for example, saline solution or the like, can be supplied into the body cavity from the liquid-supplying path. By doing so, it is possible to facilitate propagation of the ultrasonic vibration of the rod-like member to fat, and thus, it is possible to enhance the efficiency of emulsifying fat.

The above-described invention may be provided with a vibration detection electrode that detects a vibration of the columnar member and a frequency control portion that changes a frequency of the AC voltage applied by the voltage applying portion so that an amplitude value of the vibration detected by the vibration detection electrode reaches an amplitude value set in advance.

By employing such a configuration, the vibration of the columnar member is detected by the vibration detection electrode, and the frequency of the AC voltage applied by the voltage applying portion is changed so that the amplitude value of the detected vibration reaches the amplitude value set in advance. By doing so, even when a load fluctuation occurs in the vibration amplitude, the amplitude value of the bending vibration in the columnar member, that is, the amplitude value of the ultrasonic vibration in the rod-like member, can be maintained constant, which makes it possible to achieve stable melting of fat.

In the above-described invention, the piezoelectric element may be a laminated piezoelectric element in which a plurality of piezoelectric elements are laminated.

By employing the laminated piezoelectric element as the piezoelectric element, the driving voltage can be reduced by an amount substantially corresponding to the reciprocal of the number of laminated layers. For example, when a laminated piezoelectric element having a three-layer structure is employed, the driving voltage can be reduced to ⅓.

### Advantageous Effects of Invention

The present invention affords an advantage in that it is possible to efficiently remove fat by generating vibrations not only in the direction parallel to a probe but also in the direction orthogonal to the probe.

### BRIEF DESCRIPTION OF DRAWINGS

FIG. 1 is a diagram showing the overall configuration of an ultrasonic surgical device according to a first embodiment of the present invention.

FIG. 2 is a top view of a transducer in FIG. 1.

FIG. 3 is a side view of the transducer in FIG. 1.

FIG. 4 is a diagram showing the external appearance of a piezoelectric element in FIGS. 2 and 3.

FIG. 5 is a top view showing relevant portions of the ultrasonic surgical device in FIG. 1.

FIG. 6 is a cross-sectional view taken along A-A″ in FIG. 5.

FIG. 7 is a diagram showing a rotational movement at an antinode when the transducer in FIG. 1 is operated.

FIG. 8 is a diagram showing bending vibrations in an XZ plane when the transducer in FIG. 1 is operated.

FIG. 9 is a diagram showing bending vibrations in a YZ plane when the transducer in FIG. 1 is operated.

FIG. 10 is a diagram for explaining the operational effect of the ultrasonic surgical device in FIG. 1.

FIG. 11 is a diagram for explaining the operational effect of the ultrasonic surgical device in FIG. 1.

FIG. 12 is a top view of a transducer according to a first modification.

FIG. 13 is a side view of the transducer in FIG. 12.

FIG. 14 is a top view of a transducer according to a second modification.

FIG. 15 is a side view of the transducer in FIG. 14.

FIG. 16 is a diagram showing bending vibrations in an XZ plane when a transducer according to a third modification is operated.

FIG. 17 is a diagram showing the external appearance of a piezoelectric element in FIG. 16.

FIG. 18 is a top view of a transducer according to a fourth modification.

FIG. 19 is a side view of the transducer in FIG. 18.

FIG. 20 is a top view of a transducer according to a fifth modification.

FIG. 21 is a side view of the transducer in FIG. 20.

FIG. 22 is a top view of a transducer according to a second embodiment of the present invention.

FIG. 23 is a side view of the transducer in FIG. 22.

FIG. 24 is a cross-sectional view showing relevant portions of an ultrasonic surgical device according to the second embodiment of the present invention.

FIG. 25 is a top view of a transducer according to a sixth modification.

FIG. 26 is a side view of the transducer in FIG. 25.

FIG. 27 is a longitudinal sectional view in which a rod-like contactor in FIG. 26 is partially enlarged.

FIG. 28 is a diagram showing the external appearance of a piezoelectric element according to a third embodiment of the present invention.

FIG. 29 is a diagram showing the overall configuration of an ultrasonic surgical device according to the third embodiment of the present invention.

FIG. 30 is a flow chart showing processing executed by the ultrasonic surgical device in FIG. 29.

FIG. 31 is a graph for explaining the operational effect of the ultrasonic surgical device in FIG. 29.

FIG. 32 is a diagram showing the external appearance of a piezoelectric element according to a fourth embodiment of the present invention.

FIG. 33 is an exploded view of the piezoelectric element in FIG. 32.

FIG. 34 is a cross-sectional view of the piezoelectric element taken along A-A″ in FIG. 32.

### DESCRIPTION OF EMBODIMENTS

### First Embodiment

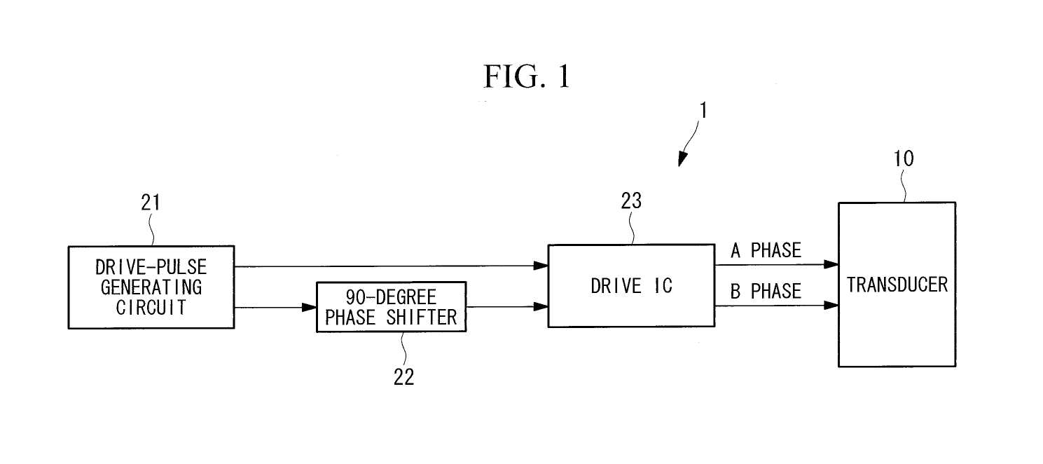

A first embodiment of the present invention will be described below by using FIGS. 1 to 21. In the following, an example in which an ultrasonic vibration device according to the present invention is applied to an ultrasonic surgical device for removing fat in a body cavity will be described.

As shown in FIG. 1, an ultrasonic surgical device 1 according to this embodiment is provided with a transducer 10 that is inserted into a body cavity, a drive-pulse generating circuit (voltage applying portion) 21 that generates drive pulses, a 90-degree phase shifter 22 that changes the phase of the drive pulses from the drive-pulse generating circuit 21, and a drive IC 23 that amplifies the drive pulses and outputs them to the transducer 10.

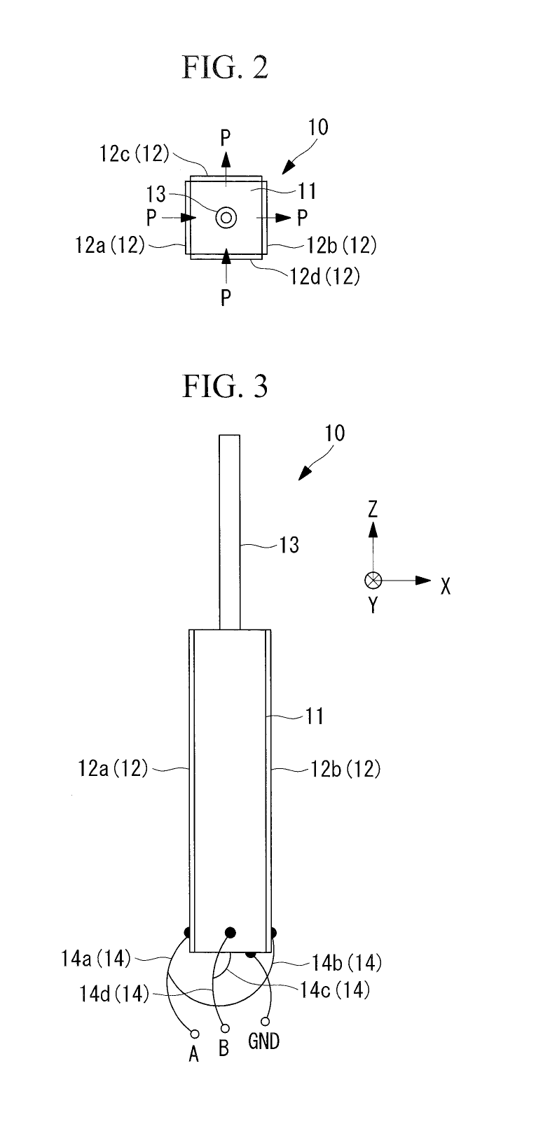

FIGS. 2 and 3 show the transducer 10 employed in this embodiment. FIG. 2 is a top view of the transducer 10 and

FIG. 3 is a side view of the transducer 10.

As shown in FIGS. 2 and 3, the transducer 10 is provided with a polygonal columnar elastic body (columnar member) 11 formed of an elastic body, piezoelectric elements 12 that are secured to four side surfaces of the polygonal columnar elastic body 11, each of which is polarized in the plate-thickness direction, and a rod-like contactor (rod-like member) 13 that is secured to an end portion of the polygonal columnar elastic body 11 and that has a smaller diameter than the polygonal columnar elastic body 11.

The material for the polygonal columnar elastic body 11 is formed of a component having a large Q value, such as a titanium alloy, stainless steel, or the like. The plate-like piezoelectric elements 12 are bonded to the four side surfaces of the polygonal columnar elastic body 11 by using epoxy resin. A hole is provided at the top end of the polygonal columnar elastic body 11, into which the rod-like contactor 13 is inserted and secured by means of press fitting or bonding.

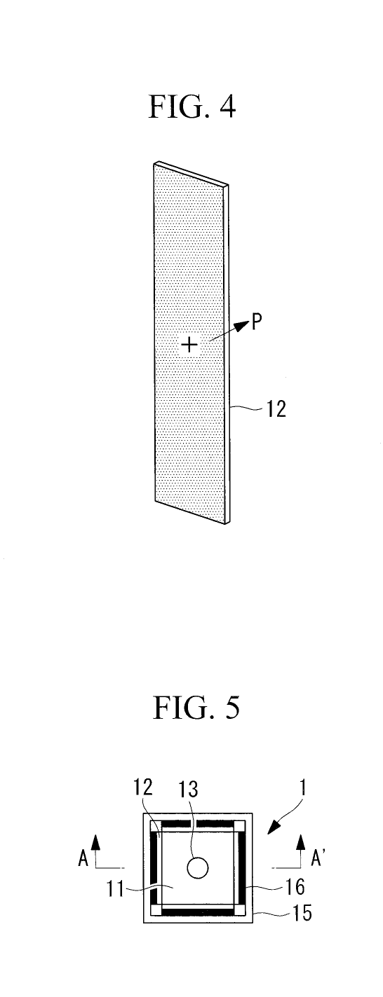

FIG. 4 shows the external appearance of the piezoelectric elements 12. The raw material for the piezoelectric elements 12 is lead zirconate titanate (PZT). The piezoelectric elements 12 have a rectangular plate shape provided with electrodes on the front surface and the back surface thereof and are polarized in the plate-thickness direction. As shown in FIG. 4, the polarization direction is shown by a polarization vector P, and the polarization vector P is a vector directed toward the negative surface (back surface) from the positive surface (front surface). These piezoelectric elements 12 are attached on the four side surfaces, paying attention to the polarization directions, so that the polarization vectors P point in the same directions at opposing surfaces of the polygonal columnar elastic body 11, as shown in FIG. 2.

As shown in FIG. 3, leads 14 for applying AC voltages to the piezoelectric elements 12 are attached to the electrode surfaces of the piezoelectric elements 12 by means of conductive adhesive or soldering. Leads 14a and 14b of a pair of piezoelectric elements 12a and 12b for exciting vibrations in the X direction are joined with each other, thus forming an A terminal. Leads 14c and 14d of a pair of piezoelectric elements 12c and 12d for exciting vibrations in the Y direction are joined with each other, thus forming a B terminal. A GND terminal that serves as a shared electrode is attached to the bottom surface of the polygonal columnar elastic body 11 by means of conductive adhesive.

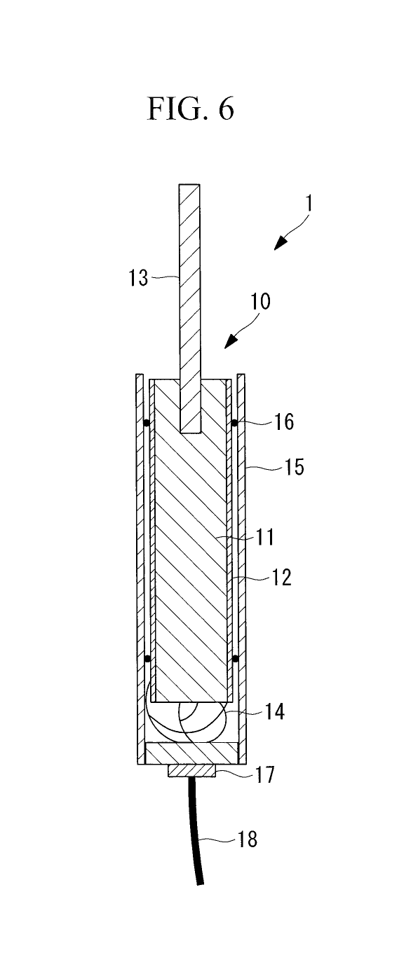

FIGS. 5 and 6 show relevant portions of the ultrasonic surgical device 1 according to this embodiment. FIG. 5 is a top view of the relevant portions of the ultrasonic surgical device 1, and FIG. 6 shows a cross-sectional view taken along A-A′ in FIG. 5.

As shown in FIGS. 5 and 6, a case 15 having a polygonal shape is provided outside the transducer 10 so as to enclose the transducer 10. Rubber pieces (holding members) 16 are provided between the transducer 10 and the case 15 near nodes of the transducer 10, described later. In other words, the transducer 10 is held in the case 15 by means of the rubber pieces 16. By holding the transducer 10 near the nodes, it is possible to prevent the vibrational energy from escaping outside the case 15 and so forth.

In addition, a connector 17 for the leads 14 is provided at the bottom surface of the case 15. A holding wire 18 is connected to the connector 17. Although not shown in the drawings, the leads 14 are contained inside the holding wire 18. In addition, the holding wire 18 serves the dual purpose of holding and manipulating the case 15 (and the transducer 10 in the interior thereof).

As shown in FIG. 1, the drive-pulse generating circuit 21 outputs two sets of drive pulses whose frequencies correspond to a predetermined bending resonance frequency. By doing so, the drive-pulse generating circuit 21 generates bending vibrations in the polygonal columnar elastic body 11 by applying AC voltages in the plate-thickness direction of the piezoelectric elements 12 via the leads 14, thus generating ultrasonic vibrations in the rod-like contactor 13. Note that the details of the operation involved when generating the bending vibrations in the polygonal columnar elastic body 11 will be described later.

Of the two sets of drive pulses output from the drive-pulse generating circuit 21, the 90-degree phase shifter 22 shifts the phase of one set of the drive pulses by 90°.

The drive IC 23 amplifies the drive pulses from the drive-pulse generating circuit 21 and the drive pulses whose phase has been shifted by 90° by the 90-degree phase shifter 22 and outputs them to the transducer 10.

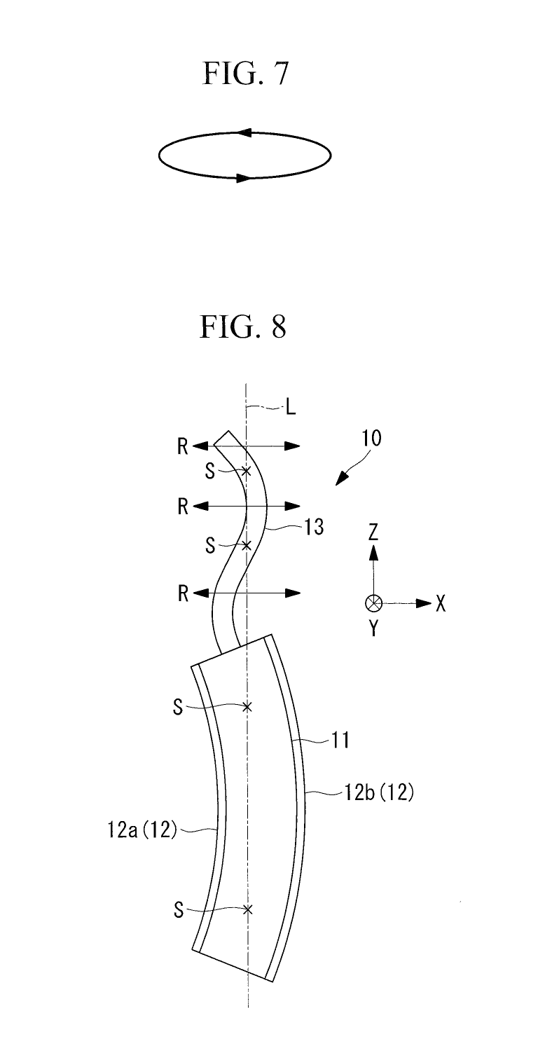

By individually applying the two sets of drive pulses that have been amplified by the drive IC 23 and whose phases have been shifted by 90°, as described above, to an A phase and a B phase of the transducer 10, it is possible to bring about rotational motion at antinodes of the transducer 10 (rod-like contactor 13), as shown in FIG. 7.

The operational effect of the ultrasonic surgical device 1 according to this embodiment, having the above-described configuration, will be described below.

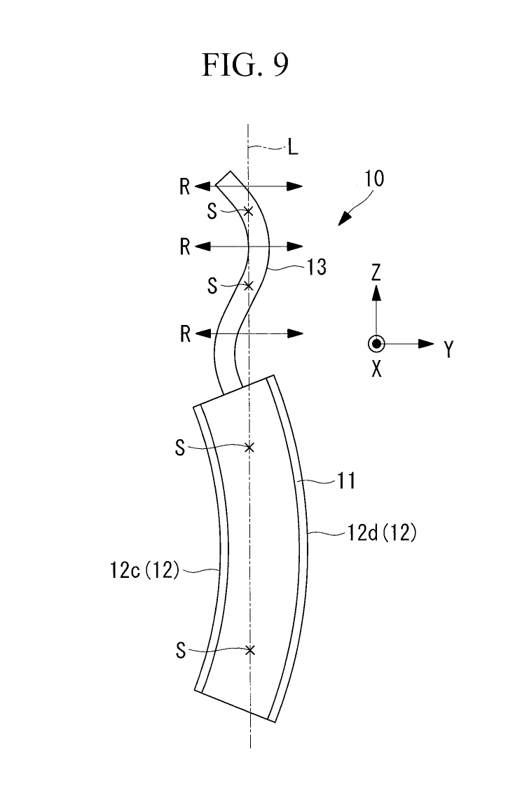

First, the operation of the transducer 10 will be described by using FIGS. 8 and 9.

As shown in FIG. 8, when an AC voltage is applied between the A terminal and the GND terminal, because the orientations of the polarizations differ between the piezoelectric elements 12 at opposing surfaces, an expanding force is generated at one surface (piezoelectric element 12b in FIG. 8) and a contracting force is generated at the other surface (piezoelectric element 12a in FIG. 8), thus generating forces that bend the polygonal columnar elastic body 11 and the rod-like contactor 13 connected thereto.

Although bending resonance modes of lower order and higher order exist, a mode shown in FIG. 8 is a mode in which nodes S exist at a total of four locations, that is, two locations in the polygonal columnar elastic body 11 and two locations in the rod-like contactor 13. Focusing on the rod-like contactor 13, antinodes R of vibrations exist at three locations, where vibrations are generated in the direction orthogonal to an axis L of the rod-like contactor 13. These vibrations are bending vibrations in an XZ plane.

The situation shown in FIG. 9 is a case in which an AC voltage is applied between the B terminal and the GND terminal, where vibrations are generated in a YZ plane in a similar manner to the bending vibrations in the XZ plane described above. Furthermore, by applying AC voltages to the A phase and the B phase at the same time, the vibrations are combined, which makes it possible to generate vibrations of an even greater magnitude. In addition, by making the phase difference thereof 90°, it is possible to cause rotational motion in the rod-like contactor 13, as shown in FIG. 7, instead of simple reciprocating vibrations.

Control of the drive circuit in this case will be described below.

As shown in FIG. 1, the two sets of drive pulses whose frequencies correspond to the predetermined bending resonance frequency are output from the drive-pulse generating circuit 21. Of the two sets of drive pulses, the phase of one set of drive pulses is shifted by 90° by the 90-degree phase shifter 22. Then, these two sets of drive pulses are amplified by the drive IC 23. Signals amplified by the drive IC 23 are applied to the A phase and the B phase, respectively, thus causing rotational motion at the antinodes R in the transducer 10 (rod-like contactor 13), as shown in FIG. 7.

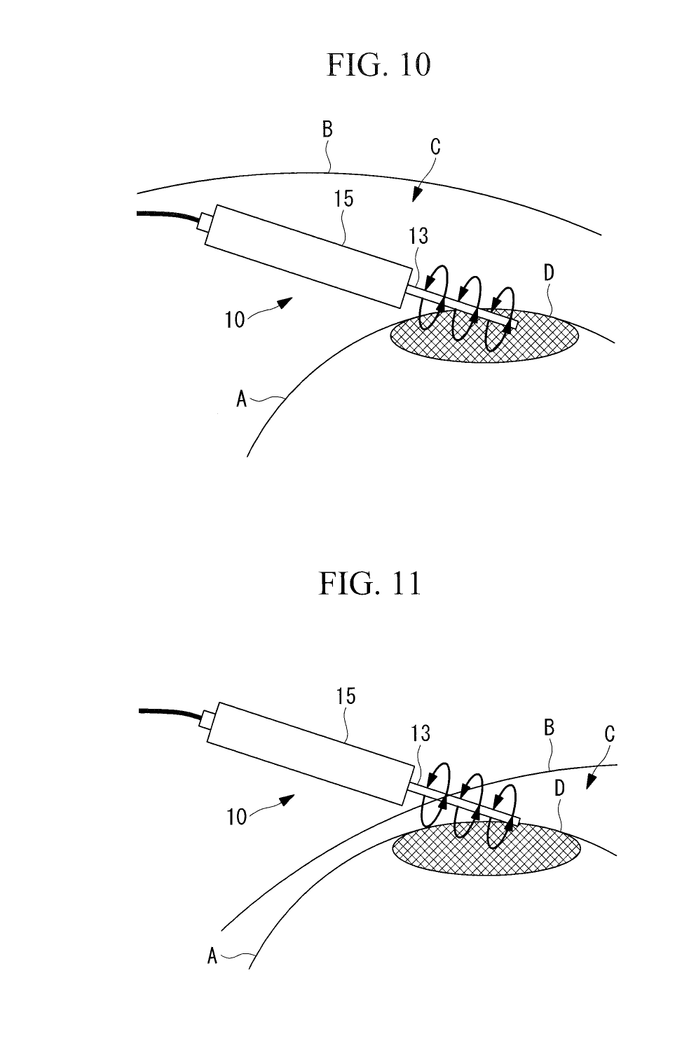

The operational effects of the ultrasonic surgical device 1 that is provided with the transducer 10 operated as described above will be described by using FIGS. 10 and 11.

In FIG. 10, the transducer 10 is inserted into the pericardial cavity C, which is a space between the pericardium B and the epicardium (a membrane at an outer surface of the heart) A via a sheath or the like. In general, myocardial infarction or the like is caused by fat D adhered to the cardiac-muscle surface. By coming into contact with the fat D, the rod-like contactor 13 exhibiting rotational bending vibrations can melt (emulsify) the fat D by means of the ultrasonic vibrations. Note that, as shown in FIG. 11, energy treatment (fat removal) may be performed by inserting only the rod-like contactor 13 into the pericardial cavity C.

As described above, with the ultrasonic surgical device 1 according to this embodiment, bending vibrations are generated in the polygonal columnar elastic body 11 by applying AC voltages in the plate-thickness direction of the piezoelectric elements 12 by means of the drive-pulse generating circuit 21. These vibrations are transmitted to the rod-like contactor 13, which is secured to the end portion of the polygonal columnar elastic body 11, thus generating ultrasonic vibrations in the rod-like contactor 13. By inserting the rod-like contactor 13 exhibiting such ultrasonic vibrations into, for example, a body cavity such as the pericardial cavity or the like, and by bringing the rod-like contactor 13 into contact with fat adhered to an inner wall of the body cavity, it is possible to melt (emulsify) the fat by means of the ultrasonic vibrations.

In this case, with the ultrasonic surgical device 1 according to this embodiment, bending vibrations in the polygonal columnar elastic body 11, in other words, vibrations transmitted to the rod-like contactor 13, are vibrations in the direction orthogonal to the axis of the rod-like contactor 13. Therefore, in the state in which the rod-like contactor 13 is inserted into the body cavity, it is possible to melt fat by means of the ultrasonic vibrations not only at the distal-end surface of the rod-like contactor 13 but also at side surfaces thereof, and thus, it is possible to efficiently melt the fat adhered to the inner wall of the body cavity.

### First Modification

A first modification of the ultrasonic surgical device according to this embodiment will be described below. Note that, in the following, for ultrasonic surgical devices according to individual modifications, the same reference signs will be assigned to commonalities with the embodiment described above, omitting descriptions thereof, and differences will mainly be described.

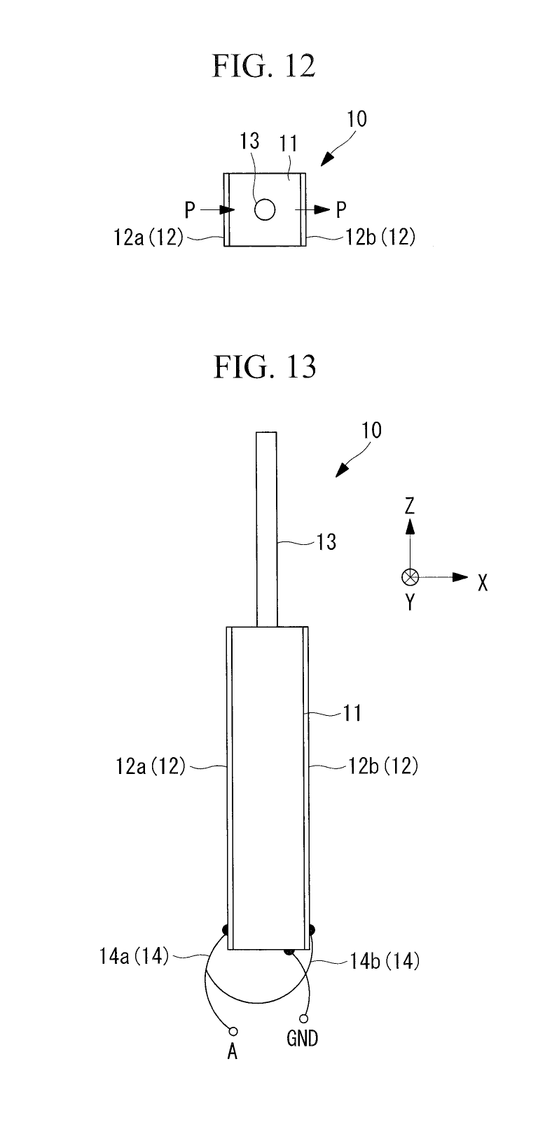

Although the piezoelectric elements 12 are bonded to the four side surfaces of the polygonal columnar elastic body 11 in the embodiment described above, as the first modification of this embodiment, by attaching the pair of piezoelectric elements 12a and 12b to two opposing side surfaces of the polygonal columnar elastic body 11, as shown in FIGS. 12 and 13, it is possible to achieve a size reduction of the transducer 10. Note that the pair of piezoelectric elements 12c and 12d may be attached to the two opposing side surfaces of the polygonal columnar elastic body 11.

### Second Modification

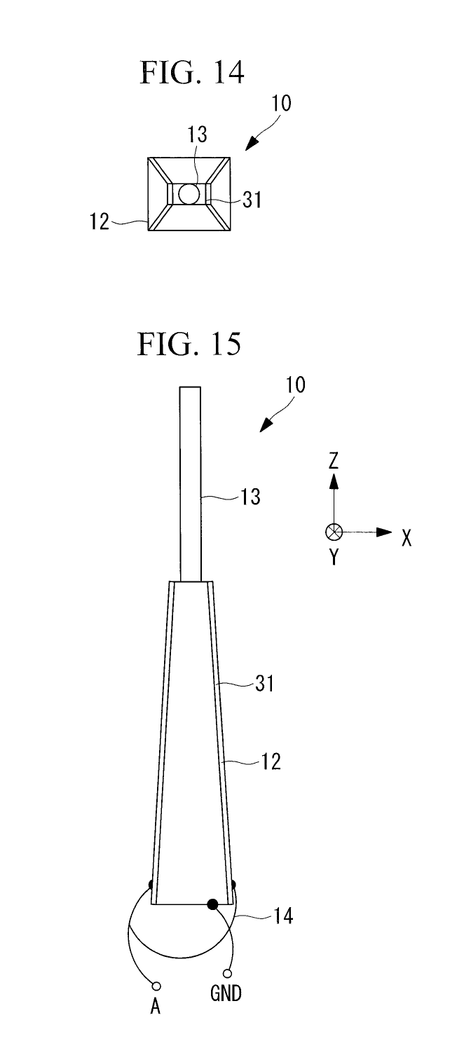

Although the polygonal columnar elastic body 11 is employed as the elastic body (columnar member) in the embodiment described above, as a second modification of this embodiment, a pyramidal elastic body 31, whose lateral cross-sectional area gradually decreases toward the distal end, as shown in FIGS. 14 and 15, may be employed. In this case, piezoelectric elements having a trapezoidal planar shape are employed as the piezoelectric elements 12.

With the ultrasonic surgical device according to this modification, it is possible to make the mechanical impedances of the rod-like contactor 13 and the pyramidal elastic body 31 similar to each other in the vicinity of a connecting position between the rod-like contactor 13 and the pyramidal elastic body 31. Because good mechanical impedance matching can be achieved in this way, the vibrational energy of the pyramidal elastic body 31 can be transmitted to the rod-like contactor 13 more efficiently.

In addition, by employing the pyramidal elastic body 31, it is possible to increase the lateral cross-sectional area of the pyramidal elastic body 31 at the proximal end thereof. By doing so, large surface areas can be ensured for the piezoelectric elements 12 to be attached to the side surfaces of the pyramidal elastic body 31, and thus, the vibrational energy generated at the pyramidal elastic body 31 can be increased.

In addition, because it is possible to make the distal end of the transducer 10 smaller, it is possible to enhance the ease of insertion into a body cavity and so forth, thus improving the usability of the device.

### Third Modification

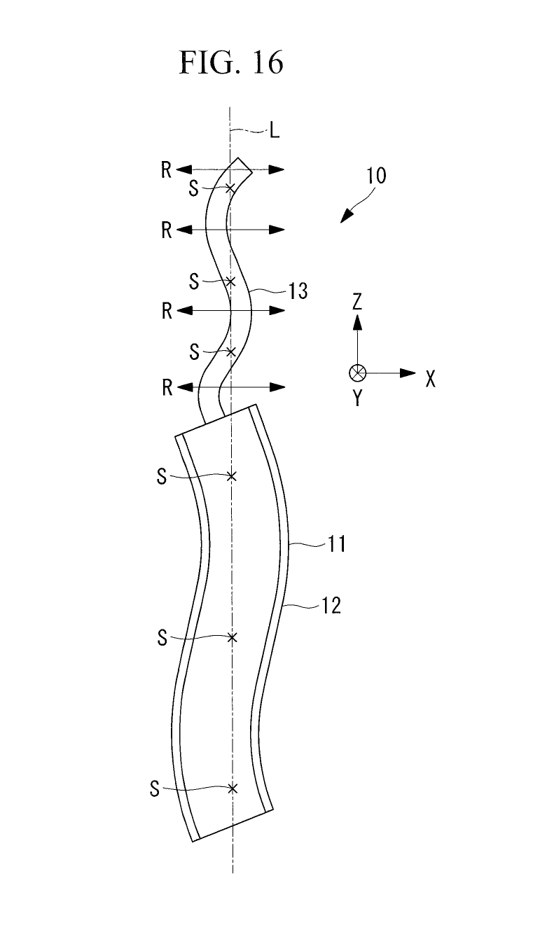

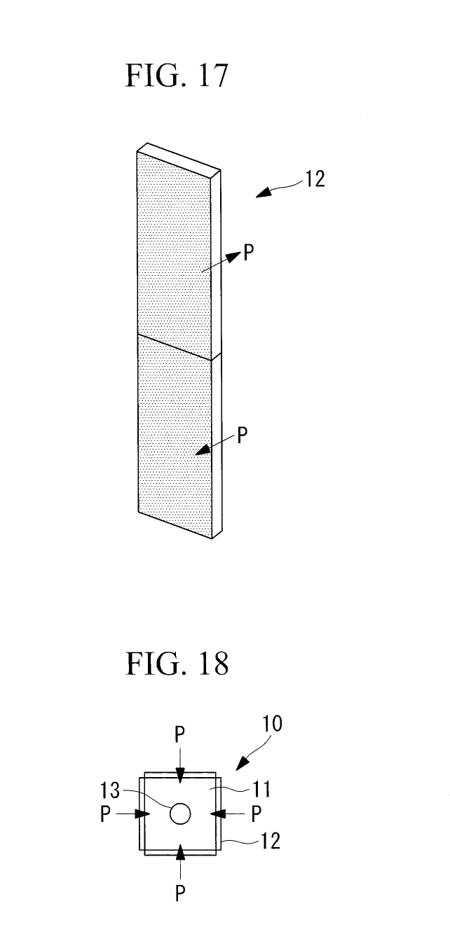

As shown in FIG. 16, as a third modification of this embodiment, a mode of an even higher order, such as one in which three nodes S exist in the polygonal columnar elastic body 11 and three nodes S (four antinodes R) exist in the rod-like contactor 13, may be employed. In this case, for example, it is necessary to make the polarization directions (orientations of the polarization vectors P) of a piezoelectric element 12 opposite each other on either side of a center portion thereof, as shown in FIG. 17.

With the ultrasonic surgical device according to this modification, it is possible to increase the number of antinodes R in the rod-like contactor 13, in other words, the positions at which the amplitude becomes the highest, and thus, it is possible to melt the fat D adhered to a cardiac-muscle surface more efficiently.

### Fourth Modification

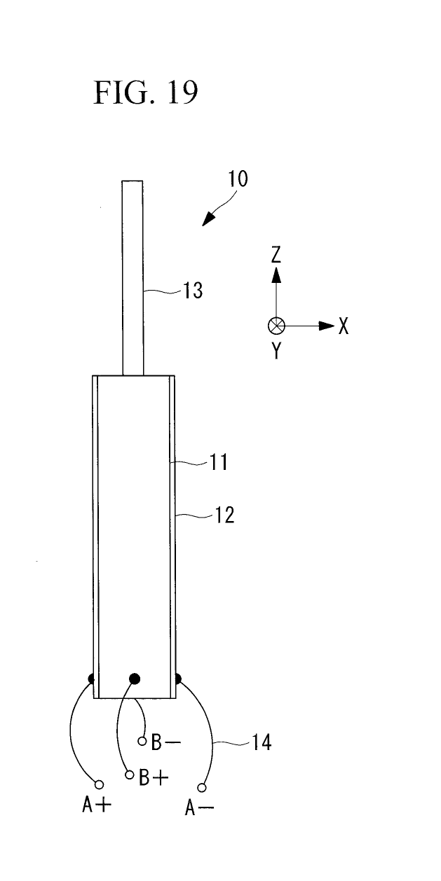

FIGS. 18 and 19 show a fourth modification of this embodiment. FIG. 18 is a top view of the transducer 10 according to this modification, and FIG. 19 is a side view of the transducer 10 according to this modification. In the case of this modification, the piezoelectric elements 12 are bonded to the polygonal columnar elastic body 11 so that the orientations of the polarization (orientations of the polarization vectors P) of the opposing piezoelectric elements 12 become opposite to each other.

In addition, regarding the leads 14, the common GND line is eliminated, and the pair of opposing piezoelectric elements 12 are driven by using one of the pair as an A+ (B+) terminal and the other as an A− (B−) terminal. By doing so, because the pair of opposing piezoelectric elements 12 are made to expand/contract in opposite phases to each other, it is possible to excite bending vibrations in the polygonal columnar elastic body 11. In other words, with the ultrasonic surgical device according to this modification, the number of leads 14 can be reduced.

### Fifth Modification

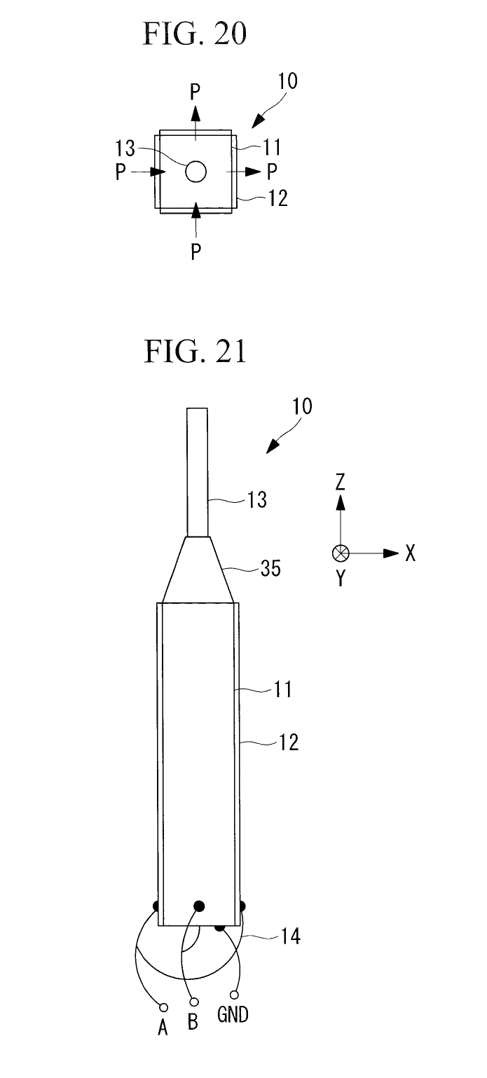

FIGS. 20 and 21 show a fifth modification of this embodiment.

In this modification, a cone-shaped horn member 35, whose the lateral cross-sectional area gradually decreases toward the rod-like contactor 13 from the polygonal columnar elastic body 11, is provided between the polygonal columnar elastic body 11 and the rod-like contactor 13. By inserting such a horn member 35, it is possible to match the mechanical impedances between the polygonal columnar elastic body 11 and the rod-like contactor 13, and thus, it is possible to increase the amplitude at the rod-like contactor 13.

### Second Embodiment

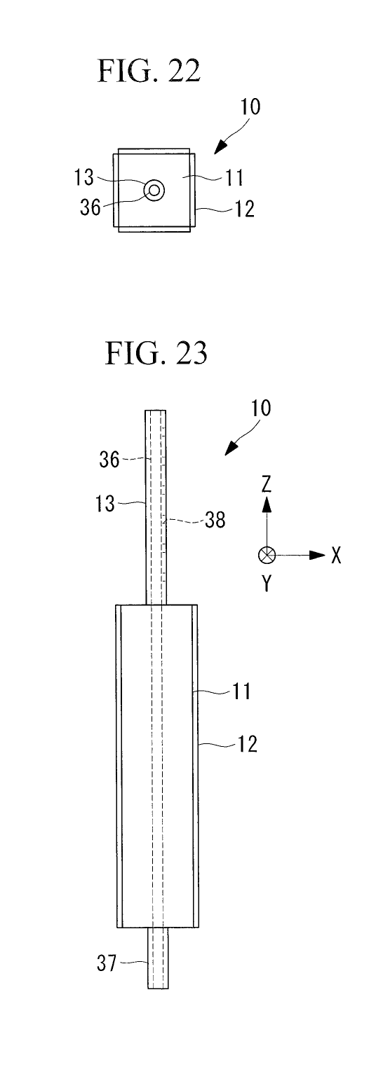

Next, an ultrasonic surgical device 2 according to a second embodiment of the present invention will be described with reference to FIGS. 22 to 27. In the following, for ultrasonic surgical devices according to individual embodiments, the same reference signs will be assigned to commonalities with the embodiment described above, omitting descriptions thereof, and differences will mainly be described.

FIG. 22 shows a top view of the transducer 10 according to this embodiment and FIG. 23 shows a side view thereof. A rear-end protrusion 37 is provided at the bottom end of the polygonal columnar elastic body 11. In addition, the rod-like contactor 13, the polygonal columnar elastic body 11, and the rear-end protrusion 37 are provided with a through-hole (suction path) 36 that passes through them in the axial direction thereof without interruption. Furthermore, a plurality of side holes 38 that communicate with the through-hole 36 are provided in a side surface of the rod-like contactor 13.

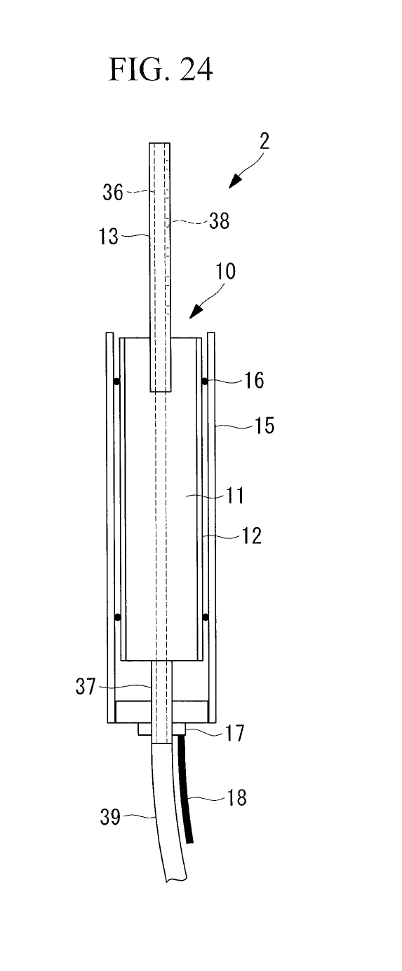

FIG. 24 is a longitudinal sectional view of relevant portions of the ultrasonic surgical device 2 according to this embodiment. The rear-end protrusion 37 is connected to a suction hose 39 at the connector 17, and the through-hole 36 communicates with the suction hose 39. Note that the suction hose 39 and the holding wire 18 extend in a bundled state. Note that, although not illustrated, a suction pump is provided at the other end of the suction hose 39.

Next, the operation of the ultrasonic surgical device 2 according to this embodiment will be described.

First, as with the embodiment described above, the relevant portions of the ultrasonic surgical device 2 according to this embodiment are inserted into the pericardial cavity C, which is a space between the pericardium B and the epicardium A, via a sheath or the like (see FIG. 10). When AC voltages are applied in this state in the plate-thickness direction of the piezoelectric elements 12 by means of the drive-pulse generating circuit 21, bending vibrations are generated in the polygonal columnar elastic body 11, and thus, ultrasonic vibrations are generated in the rod-like contactor 13 connected to the distal end of the polygonal columnar elastic body 11.

By coming into contact with the fat D, the rod-like contactor 13 exhibiting ultrasonic vibrations can emulsify the fat D. The fat D emulsified at the rod-like contactor 13 is sucked from the through-hole 36 provided at the distal-end surface of the rod-like contactor 13 or the side holes 38 provided at the side surface thereof by means of the suction hose 39 and is externally discharged.

As described above, with the ultrasonic surgical device 2 according to this embodiment, in addition to the same advantages as those afforded by the above-described embodiment, it is possible to discharge the fat component emulsified by the ultrasonic vibrations of the rod-like contactor 13 outside the body. Note that, although the side holes 38 in this embodiment are provided only in the X-direction for the convenience of preparing the drawings, they may be provided in the Y-direction, and it is desirable that the side holes 38 be provided in multiple directions in a radiating manner.

### Sixth Modification

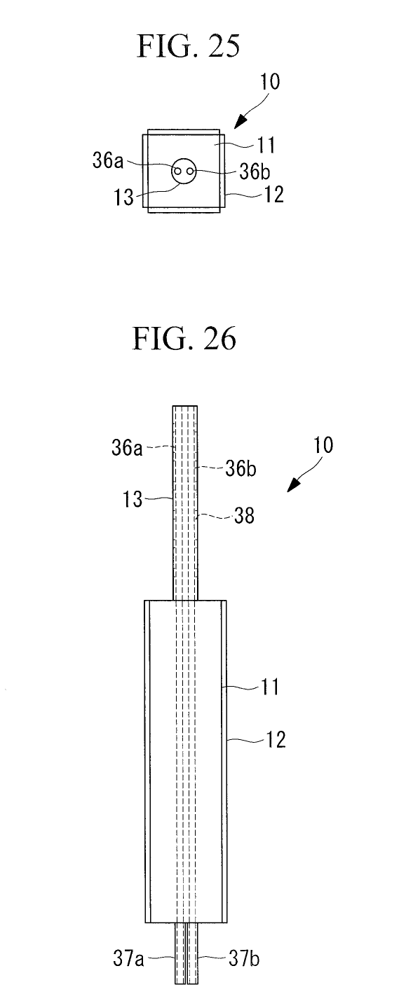

FIGS. 25 to 27 show a sixth modification of this embodiment.

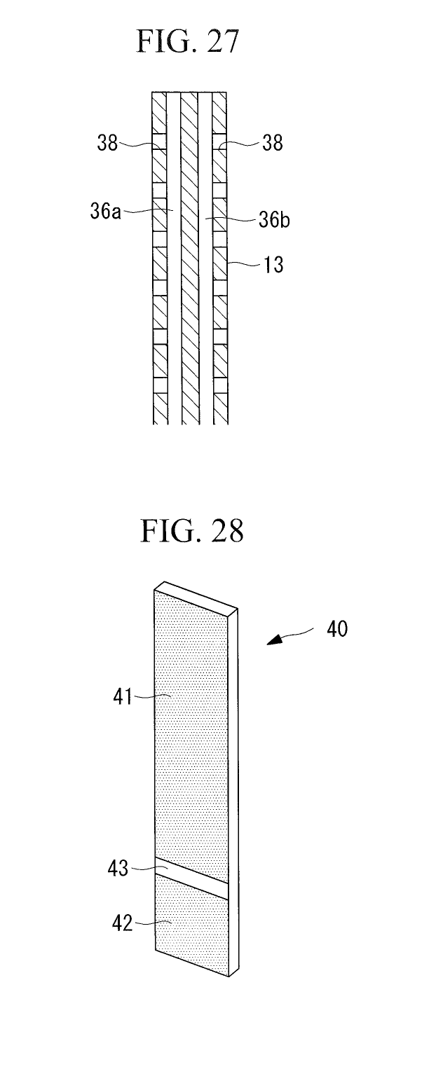

As shown in FIGS. 25 and 26, in this modification, a liquid-supplying through-hole (liquid-supplying path) 36a and a suction through-hole (suction path) 36b are independently provided as the through-holes. A rear-end liquid-supplying protrusion 37a and a rear-end suction protrusion 37b are provided at the bottom surface of the polygonal columnar elastic body 11. Furthermore, as shown in FIG. 27, side holes 38 that individually communicate with the liquid-supplying through-hole 36a and the suction through-hole 36b are provided in the side surface of the rod-like contactor 13.

With the ultrasonic surgical device according to this modification, liquid, such as saline solution or the like, can be supplied into the body cavity via the liquid-supplying through-hole 36a, and the liquid (saline solution) can reliably be interposed between a fat portion and the rod-like contactor 13. By doing so, it is possible to facilitate propagation of ultrasonic vibrations to the fat, which makes it possible to enhance the efficiency of emulsifying fat. Note that, although the side holes 38 in this modification are provided only in the X-direction for the convenience of preparing the drawings, they may be provided in the Y-direction, and it is desirable that the side holes 38 be provided in multiple directions in a radiating manner.

### Third Embodiment

Next, an ultrasonic surgical device 3 according to a third embodiment of the present invention will be described with reference to FIGS. 28 to 31.

FIG. 28 shows a piezoelectric element 40 employed in this embodiment. A feature of this piezoelectric element 40 is that an electrode thereof is divided into two by an insulating region 43. The upper portion serves as a drive electrode 41, and the lower portion serves as a vibration detection electrode 42. It suffices that this piezoelectric element 40 be provided at least one location among the piezoelectric elements 12 attached on the four side surfaces of the polygonal columnar elastic body 11. Note that, in the case in which the piezoelectric elements 40 are provided at multiple locations, the output therefrom should be connected in parallel.

Next, the operation of the ultrasonic surgical device 3 according to this embodiment will be described.

Although the piezoelectric element 40 is deformed when a voltage is applied thereto (converse piezoelectric effect), the deformation generates a voltage (piezoelectric effect). Therefore, by observing the voltage of a vibration detection electrode 42, it is possible to detect AC voltages proportional to the magnitude of the vibrations.

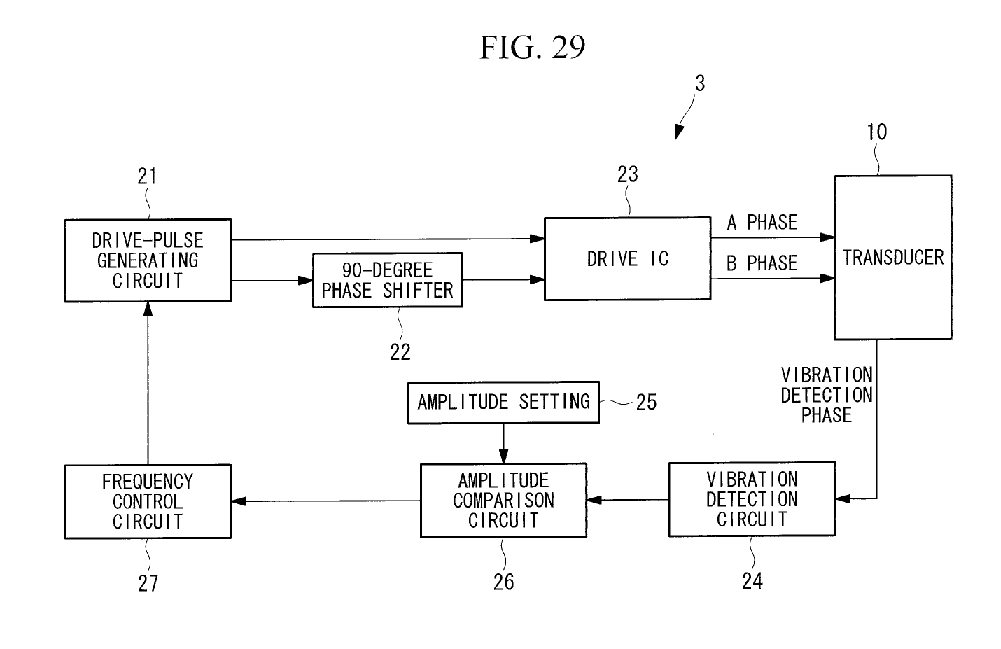

FIG. 29 shows a drive circuit employing the vibration detection electrode 42 (vibration detection phase).

AC drive pulses having an initial frequency value are output from the drive-pulse generating circuit 21, and drive pulses in the B phase are converted by the 90-degree phase shifter 22 into signals whose phase differs by 90°. Drive pulses in the A phase from the drive-pulse generating circuit 21 and the drive pulses in the B phase from the 90-degree phase shifter 22 are amplified by the drive IC 23. These amplified drive pulses are applied to the A phase and the B phase of the transducer 10.

When the transducer 10 is vibrated, AC voltages are output from the vibration detection phase. The signals thereof are detected by the vibration detection circuit 24, are amplified by a predetermined gain, and are output to an amplitude comparison circuit 26. An amplitude value set in advance as an amplitude setting 25 and an amplitude value from the vibration detection circuit 24 are compared at the amplitude comparison circuit 26, and a judgment signal thereof is output to a frequency control circuit (frequency control portion) 27. A frequency to be set is determined at this point, the result of which is output to the drive-pulse generating circuit 21, and the driving frequency is updated. Consequently, the drive pulses generated at the drive-pulse generating circuit 21 are always controlled to a desirable vibration amplitude value.

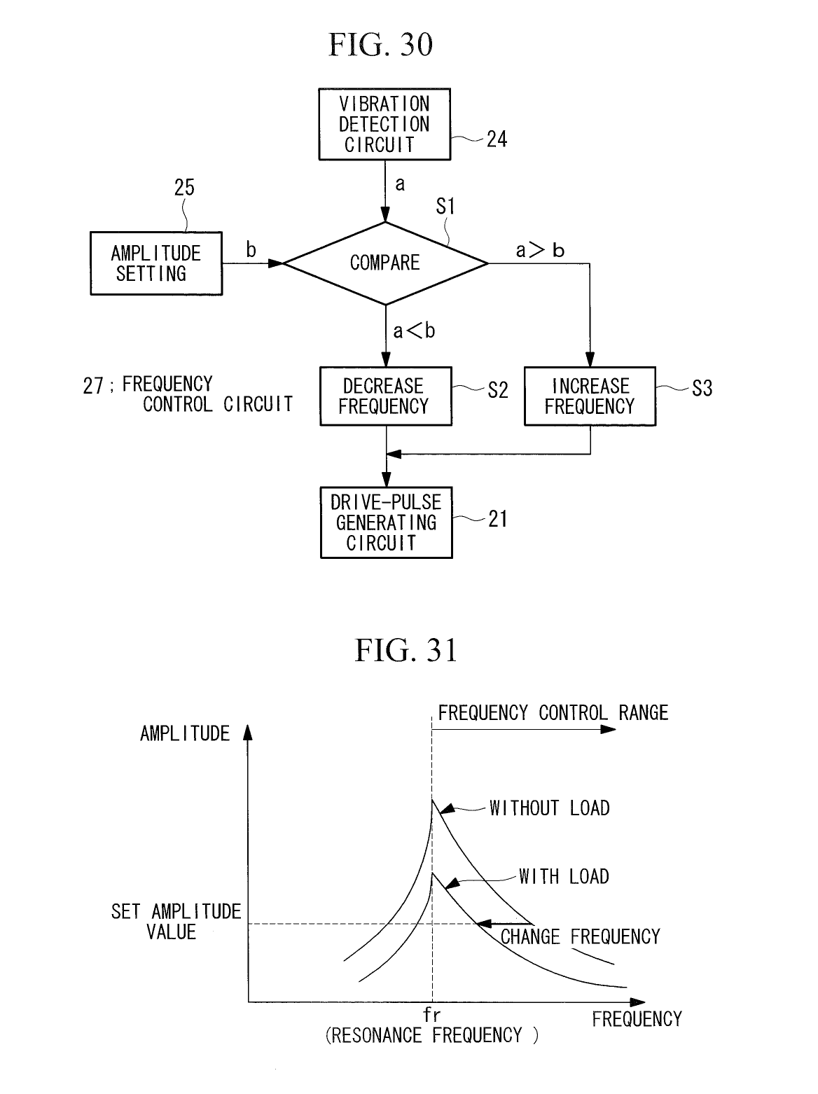

The above-described control will be described below by using the flowchart in FIG. 30.

As shown in FIG. 30, defining the amplitude value of the drive pulses detected by the vibration detection circuit 24 (hereinafter referred to as “detected amplitude value”) as a, and the amplitude value set in advance as the amplitude setting 25 (hereinafter referred to as “set amplitude value”) as b, the magnitude of the detected amplitude value a and that of the set amplitude value b are compared at the amplitude comparison circuit 26 (Step S1).

In the case in which the set amplitude value b is greater than the detected amplitude value a, the driving frequency of the drive pulses generated by the drive-pulse generating circuit 21 is decreased by means of the frequency control circuit 27 (Step S2). On the other hand, in the case in which the set amplitude value b is less than the detected amplitude value a, the driving frequency of the drive pulses generated by the drive-pulse generating circuit 21 is increased by means of the frequency control circuit 27 (Step S3).

Here, as shown in FIG. 31, the vibration amplitudes of the rod-like contactor 13 and the polygonal columnar elastic body 11 reach maximum values at a resonance frequency (fr). When a load is exerted on the rod-like contactor 13, the amplitude characteristic thereof is reduced overall. Therefore, in that case, the driving frequency is made closer to the resonance frequency so as to achieve the same amplitude. Note that, the frequency control range is set higher than the resonance frequency.

As described above, with the ultrasonic surgical device 3 according to this embodiment, by providing the vibration detection electrode 42 as an electrode of the piezoelectric element 40, by constantly detecting the vibrations of the transducer 10, and by constantly controlling the frequency so that the detected value is kept constant, the amplitude value of the polygonal columnar elastic body 11, that is, the amplitude value of the ultrasonic vibrations in the rod-like contactor 13, can be maintained constant, even when load fluctuation occurs in the vibration amplitude, which makes it possible to achieve stable melting of fat.

### Fourth Embodiment

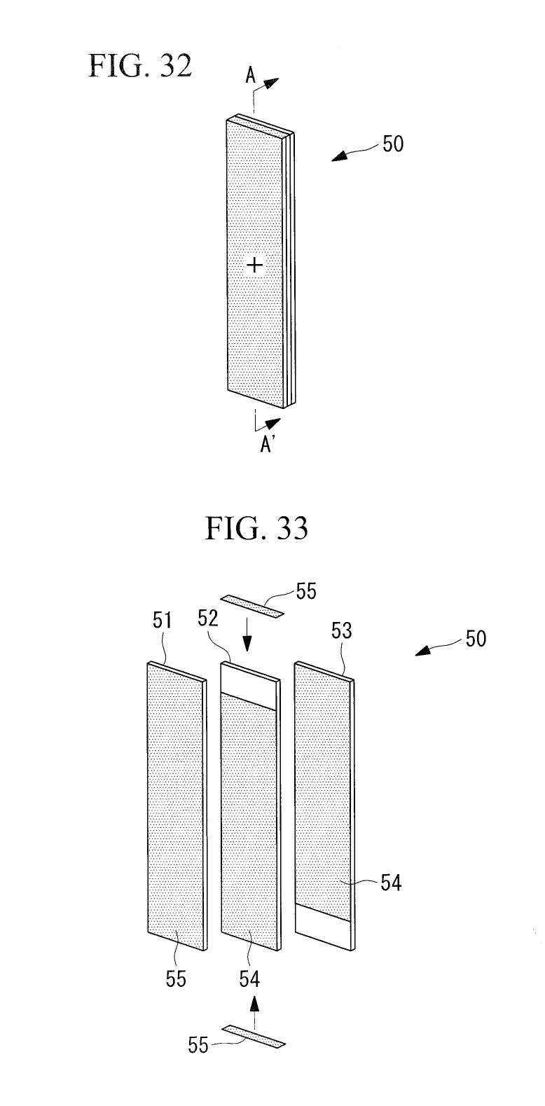

Next, an ultrasonic surgical device according to a fourth embodiment of the present invention will be described with reference to FIGS. 32 to 34.

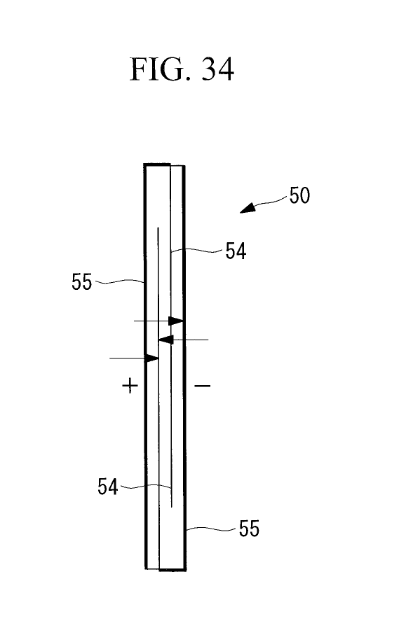

FIG. 32 shows the external appearance of a piezoelectric element 50 employed in this embodiment, FIG. 33 is an exploded view and FIG. 34 is a cross-sectional view taken along A-A′ in FIG. 32.

As shown in FIGS. 32 to 34, the piezoelectric element 50 employed in this embodiment is a piezoelectric element having a laminated structure, that is, a laminated piezoelectric element. In the laminated piezoelectric elements 50, internal electrodes (silver-palladium alloy) 54 are formed by individually providing insulating portions on portions of surfaces of piezoelectric sheets 51, 52, and 53 having a thickness of several tens of micrometers, as shown in FIG. 33. These sheets are laminated as shown in FIG. 33, followed by firing. Finally, external electrodes (silver) 55 are plated, as shown in FIG. 33.

With the ultrasonic surgical device according to this embodiment employing the laminated piezoelectric element as described above, by employing the laminated piezoelectric element, the driving voltage can be reduced by an amount substantially corresponding to the reciprocal of the number of laminated layers. In this embodiment, because a laminated piezoelectric element having a three-layer structure is employed, the driving voltage can be reduced to ⅓.

Note that, in this embodiment, the same control as that in the third embodiment described above is made possible by providing vibration detection regions in portions of the internal electrodes 54, which makes it possible to achieve stable melting of fat.

In addition, although a laminated piezoelectric element having a three-layer structure is employed in this embodiment, a laminated piezoelectric element having an N-layer structure (N is an arbitrary integer) may be employed. In this case, the driving voltage can be made 1/N.

As above, although the individual embodiments and the individual modifications of the present invention have been described in detail with reference to the drawings, specific configurations are not limited to these embodiments, and design alterations or the like within a range that does not depart from the scope of the present invention are also encompassed. For example, the present invention may be applied to embodiments in which the individual embodiments and individual modifications described above are appropriately combined.

### REFERENCE SIGNS LIST

1, 2, 3 ultrasonic surgical device

10 transducer

11 polygonal columnar elastic body (columnar member)

12 piezoelectric element

13 rod-like contactor (rod-like member)

14 lead

15 case

16 rubber piece (holding member)

21 drive-pulse generating circuit (voltage applying portion)

22 90-degree phase shifter

23 drive IC

31 pyramidal elastic body (columnar member)

36 through-hole (suction path)

36a liquid-supplying through-hole (liquid-supplying path)

36b suction through-hole (suction path)

40 piezoelectric element

50 piezoelectric element

1. An ultrasonic vibration device comprising:
a columnar member formed of an elastic body;
a piezoelectric element that is secured to a side surface of the columnar member and that is polarized in a plate-thickness direction thereof;
a rod-like member that is secured to an end portion of the columnar member and that has a smaller diameter than the columnar member; and
a voltage applying portion that generates a bending vibration in the columnar member by applying an AC voltage in the plate-thickness direction of the piezoelectric element, thus generating an ultrasonic vibration in the rod-like member.

2. An ultrasonic vibration device according to claim 1, wherein the columnar member is a polygonal columnar member.

3. An ultrasonic vibration device according to claim 1, wherein the columnar member is a pyramidal member whose a lateral cross-sectional area gradually decreases toward a connecting position with the rod-like member.

4. An ultrasonic vibration device according to claim 1,
wherein a pair of the piezoelectric elements that are disposed facing each other with the columnar member placed therebetween are provided, and
the pair of the piezoelectric elements are disposed so that orientations of the polarizations thereof point in the same direction.

5. An ultrasonic vibration device according to claim 1,
wherein a pair of the piezoelectric elements that are disposed facing each other with the columnar member placed therebetween are provided, and
the pair of the piezoelectric elements are disposed so that orientations of the polarizations thereof point in opposite directions.

6. An ultrasonic vibration device according to claim 1,
wherein a plurality of the piezoelectric elements are provided, and
the plurality of piezoelectric elements are disposed side-by-side in an axial direction of the columnar member so that the polarizations of the adjacent piezoelectric elements are oriented differently.

7. An ultrasonic vibration device according to claim 1, further comprising:
a case that accommodates the columnar member; and
a holding member that is provided between the case and the columnar member and that holds the columnar member at a node of the bending vibration.

8. An ultrasonic vibration device according to claim 1, further comprising:
a suction path for sucking tissue, provided inside the rod-like member and the columnar member.

9. An ultrasonic vibration device according to claim 8, further comprising:
a liquid-supplying path provided inside the rod-like member and the columnar member.

10. An ultrasonic vibration device according to claim 1, further comprising:
a vibration detection electrode that detects a vibration of the columnar member; and
a frequency control portion that changes a frequency of the AC voltage applied by the voltage applying portion so that an amplitude value of the vibration detected by the vibration detection electrode reaches an amplitude value set in advance.

11. An ultrasonic vibration device according to claim 1, wherein the piezoelectric element is a laminated piezoelectric element in which a plurality of piezoelectric elements are laminated.
